(19) United States
(12) Patent Application Publication
(10) Pub. No.: US 2008/0140210 A1
(43) Pub. Date: Jun. 12, 2008

(54) MODULAR SHOULDER PROSTHESIS

(76) Inventors: Robert L. Doubler, Ida, MI (US); John Hammill, Rossford, OH (US)

Correspondence Address: MCHALE & SLAVIN, P.A., 2855 PGA BLVD, PALM BEACH GARDENS, FL, 33410, US

(21) Appl. No.: 11/469,696

(22) Filed: Sep. 1, 2006

(51) Int. Cl. A61F 2/40

(52) U.S. Cl. 623/19.14; 623/22.42

(57) ABSTRACT

The instant invention provides a modular shoulder prosthesis. The prosthesis includes an intramedullary rod element which is to be inserted in a bone. The proximal portion of the rod includes a self-holding taper which is telescoped into one end of a bore in the body element. The mating tapered surfaces of the rod and the body bore form a rotationally movable connection. Whereby the body of the prosthesis may be rotationally positioned after insertion of the rod. A neck element having a shaped protrusion is telescoped into a cavity in the other end of the body bore to prevent rotational movement therebetween. A threaded fastener provides a drawing force to lock all of the components together into a fixed orientation.

### RELATED APPLICATIONS

The instant invention is related to U.S. Pat. No. 6,299,648 entitled Locking Hip Prosthesis, U.S. Pat. No. 6,355,068 entitled Sight Gauge Modular Joint and Method, U.S. Pat. No. 6,440,171 entitled Double D Locking Prosthesis, U.S. Pat. No. 6,692,530 entitled Split Sleeve Modular Joint and U.S. Pat. No. 7,033,399 entitled Welded Hip Prosthesis, the contents of which are incorporated herein by reference.

### FIELD OF INVENTION

This invention relates to the medical field of orthopaedics and joint replacement. More specifically, the instant invention is directed to a modular shoulder prosthesis which provides the ability to custom fit an artificial joint to a patient's anatomy, and most specifically to the connection between the prosthesis components which secures the components together in a manner which prevents relative movement between the components after assembly.

### BACKGROUND OF THE INVENTION

Artificial joints or prosthesis have now been constructed for almost every natural joint in the living body. As the medical field gains more understanding of the problems involved in mating inanimate constructions with animate tissue and designing mechanical devices that can duplicate natural movement, the number of implantations will continue to increase. Better engineering, accompanied with miniaturization, permits reconstruction of the major joints, such as the hip, knee, shoulder, elbow, wrist and ankle with increased reliability and range of movement. The prosthesis construction of the instant invention may be utilized in all major joints.

The natural tubercle is the broadened area offset from the end of the humerus. The natural tubercle and humeral head may be at any radial angle about the axis of the humerus. This natural angular relationship must be reproduced by the intramedullary rod and the artificial tubercle. The artificial tubercle is seated in the end of the patient's humerus and is the main load bearing element of the prosthesis. It is important that this load, which is mostly compression, is transmitted along the axis of the humerus. A spherically shaped head element is attached to the tubercle element at a fixed angle. The angle between the tubercle and the humeral head should be variable to reproduce the retroversion angle of the patient's natural joint. The humeral head carries cantilevered forces in torque and compression between the glenoid surface and the tubercle. It is important that these forces do not result in relative movement between the tubercle and the humeral head or between the tubercle and the stem.

Shoulder implant prostheses generally consist of a humeral “head” portion of the implant which articulates with the natural or artificial glenoid surface, and a “stem” portion of the implant which provides fixation within the humeral canal. In replacing a shoulder joint, the head of the humerus is removed. The end portion of the humerus is then shaped and prepared for receiving the prosthesis so that the artificial joint will closely approximate the natural shoulder. Early shoulder implants were unitary structures which combined the stem and the head. This construction required large inventories of prostheses to accommodate various sized and shaped anatomies.

Later shoulder implant designs become modular. These modular implants were characterized in that the head independently mated via “taper-lock” connection to the humeral stem. This modularity increased the options for the surgeon by offering significantly more sizes of heads, e.g. ten to twenty heads instead of the three to six heads available in earlier non-modular prostheses and more stems, e.g. five to ten instead of the two to four sizes of stems available in non-modular prostheses. By manufacturing these components with interchangeable connections but different external sizes, inventories may be smaller because of the ability to mix and match components. Also, the modular prosthesis provides more flexibility in customizing the various components of a joint to the various parts of a patient's natural joint.

In a three piece artificial shoulder joint, the stem is further broken into an intermedullary “rod” element and a “body” element. In addition to the head, these components are available in various sizes for customization by the surgeon. The rod is inserted into the end of the humerus. The rod acts as a stabilizer in maintaining the artificial joint in the axis of the humerus. The upper portion of the rod which extends out of the humerus is fitted into a body element which is shaped like the removed broad head of the humerus which it replaces. This element, along with the rod, is used to adjust the length of the prosthesis to approximate the natural length of the humerus. All these elements have a central bore and are permanently secured together by a bolt which is inserted into the body element and is threaded into the upper end of the rod.

With the advantage of flexibility gained by modular prostheses, there comes the requirement that there be no movement between the several parts or elements after implantation. These movements may cause misalignment of the joint resulting in increased pain, trauma to the joint, and even dislocation of the joint. In some cases, the intramedullary rod may be attached to the bone with bone cement while, in other cases the cement is omitted. When the cement is omitted, the placement and fixation of the intramedullary rod becomes more critical to pain free usage of the prosthesis. Further, it is most important that the intramedullary rod not be disturbed after insertion since this would corrupt the union between the rod and the interior of the humerus. In order to maintain the original union between the humerus and the intramedullary rod, modular prosthesis have been developed to allow rotational adjustment of the several parts or elements about the emplaced rod during the placement of the prosthesis to more closely reproduce the natural structure of the shoulder. It has been found that, in some cases, as the intramedullary rod is inserted into the bone canal, there is rotational movement of the rod. In order to preserve the union between the rod and the bone, there must be a mechanism to accommodate the changed angular orientation of the proximal end of the intramedullary rod so that the prosthesis closely approximates the natural tubercle and humeral head.

While the above description refers to a modular shoulder prostheses, substantially the same considerations must be given to other modular prosthesis, such as a knee prosthesis in which an intramedullary rod is placed in the lower end of the femur and in the upper end of the tibia, or the elbow in which an intramedullary rod is placed in the lower end of the humerus and the upper end of the radius or ulna, or a hip prosthesis in which an intermedullary rod is placed in to the upper end of the femur. Because of individual physical anomalies, the functional prosthesis must be capable of angular adjustment to conform to the natural physique.

While the foregoing described prior art devices have improved the art and in some instances enjoyed commercial success, there remains nonetheless a need in the art for a prosthesis that provides rotation for adjustment between the rod and body elements and a fixed connection between the body and neck elements. Such a prosthesis should include juxtapositioned surfaces between the neck and body elements for load support and transfer of load to the rod element. The prosthesis should also include a fixed connection between the neck element and the body element to eliminate rotation therebetween while still providing interchangeability of components for close approximations of various anatomies.

### SUMMARY OF THE INVENTION

In a particularly preferred embodiment of the instant invention, a modular prosthesis is taught which has an intramedullary rod element which is to be inserted in a bone. The rod has a frustoconically shaped proximal portion which is telescoped into one end of a bore in the prosthesis body element. The distal end of the rod is constructed for insertion into the intermedullary canal of the humerus. The mating surfaces of the proximal portion of the rod and the prosthesis body bore form a rotationally movable connection. A neck element having a tubular protuberance with a contoured perimeter is telescoped into the other end of the body element bore having a conjugately shaped bore until two load-bearing surfaces come into juxtaposed contact. The contoured protuberance extends outwardly from one of the load-bearing surfaces to interlock with a socket formed into the adjacent load-bearing surface to form a rotatably fixed connection. All three elements are locked together by a bolt extending through the neck and engaging the rod, forming a locked integral prosthesis. A head preferrably having a frustoconical bore for telescoping attachment to a second protuberance on the neck forms a rotationally adjustable connection. The head bore may be offset from the center portion of the head and the necks second protuberance may be supplied with various inclinations and/or offsets to facilitate interchangeability for various anatomies.

Accordingly, it is an objective of the instant invention to provide a shoulder joint with an intramedullary rod element which is connected with the body element in such a manner as to allow rotational movement between the elements after insertion of the rod. Rotational movement, in this context, refers to the turning of either element in a plane normal to the common longitudinal axis of the elements.

It is a further objective of the instant invention to provide a connection between the neck and body elements that prevents rotational movement between the elements.

It is a further objective of the instant invention to provide the intramedullary rod with a fluted exterior surface for increasing the surface area of the junction between the rod and the intramedullary canal of the humerus.

It is yet another objective of the instant invention to provide a connection between the body element and the intermedullary rod that includes locking tapers to provide a lockable connection between the elements.

It is a further objective of the invention to provide a connection between the body element and the intramedullary rod that provides a pre-set limit to the combined length of the elements.

It is a still further objective of the instant invention to provide an array of neck elements which allow for adjustment of inclination angle, height and offset.

It is an even further objective of the instant invention to provide a four piece modular shoulder joint prosthesis to provide intra-operative fit selection to build a best-fit shoulder joint.

It is still yet a further objective of the instant invention to provide a connection between the neck and body elements that includes a load-bearing surface to provide load transfer between the elements.

Still a further objective of the instant invention is to provide a neck element having a shaped tubular protuberance sized for telescoping engagement into a conjugately shaped bore formed in the proximal end of the body element bore to form a non-rotatable connection

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE FIGURES

FIG. 1 is a plan view of one embodiment of the instant invention;

FIG. 2 is a top perspective view of the embodiment shown in FIG. 1;

FIG. 3 is a section view of the embodiment shown in FIG. 1 taken along lines 3-3;

FIG. 4 is an exploded front perspective view of the embodiment shown in FIG. 1;

FIG. 5A is a top perspective view illustrating one embodiment of the body element having an interlocking contoured socket;

FIG. 5B is a bottom perspective view of one embodiment of a neck element having an interlocking contoured protrusion;

FIG. 5C is a bottom view of the neck element embodiment shown in FIG. 5B;

FIG. 6A is a top perspective view illustrating one embodiment of the body element having an interlocking contoured socket;

FIG. 6B is a bottom perspective view of one embodiment of a neck element having an interlocking contoured protrusion;

FIG. 6C is a bottom view of the neck element embodiment shown in FIG. 6B;

FIG. 7 is a top view of one embodiment of the instant invention illustrating a neck element having a posterior offset;

FIG. 8 is a side view of one embodiment of the instant invention illustrating a neck element having a medial offset;

FIG. 9 is a top view of the neck embodiment illustrated in FIG. 7;

FIG. 10 is a side view of the neck embodiment illustrated in FIG. 8;

FIG. 11 is a top perspective view of the neck embodiment illustrated in FIG. 9;

FIG. 12 is a side view illustrating a range of neck inclinations available for fitting to an anatomy;

FIG. 13 is a perspective view illustrating a series of neck embodiments each having a different inclination;

FIG. 14 is a side view of the series of neck embodiments shown in FIG. 13;

FIG. 15 is a side view of a neck element embodiment;

FIG. 16 is a perspective view illustrating assembly of the stem element and the body element;

FIG. 17 is a perspective view illustrating an assembly of a stem element and a body element;

FIG. 18 is a side view illustrating one embodiment of the stem element;

FIG. 19 is a bottom view of the stem element illustrated in FIG. 18;

FIG. 20 is a top view illustrating a shoulder prosthesis having an eccentric humeral head element;

FIG. 21 is a front plan view illustrating the embodiment shown in FIG. 20;

FIG. 22 is a bottom perspective view illustrating a humeral head element with an eccentric taper-lock bore;

FIG. 23 is a rear plan view of the embodiment shown in FIG. 21;

FIG. 24 is a section view of an alternative embodiment taken along line 3-3 of FIG. 2;

FIG. 25A is a plan view of the rod element as shown in FIG. 24;

FIG. 25B is a perspective view illustrating a nut element as shown in FIG. 24.

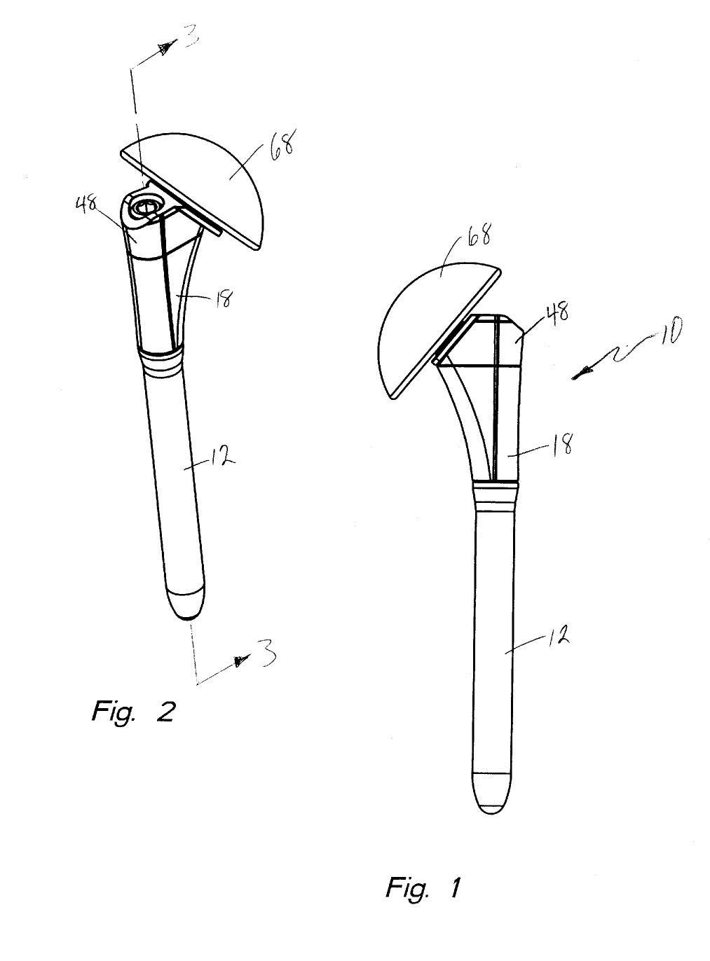

### DETAILED DESCRIPTION OF THE INVENTION

While the present invention is susceptible of embodiment in various forms, there is shown in the drawings and will hereinafter be described a presently preferred embodiment with the understanding that the present disclosure is to be considered an exemplification of the invention and is not intended to limit the invention to the specific embodiments illustrated.

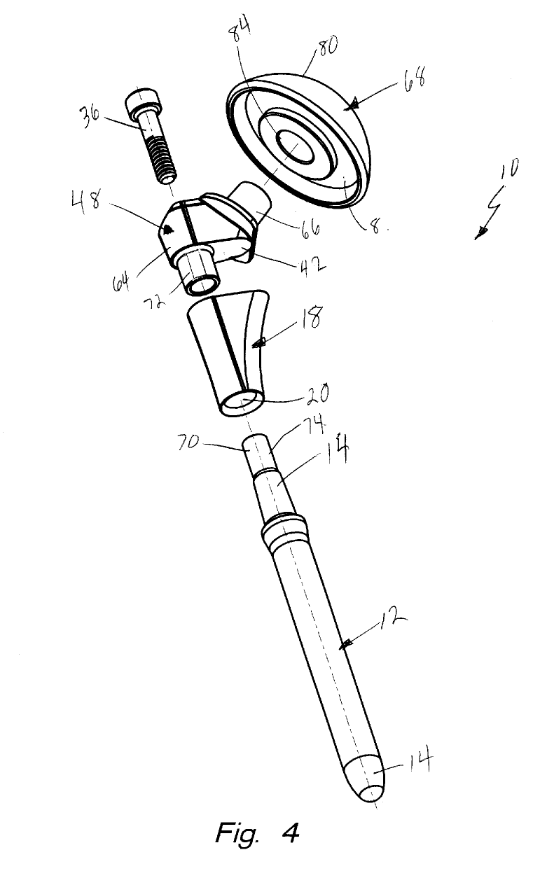

Referring to the Figures, various embodiments of the modular shoulder prosthesis 10 are illustrated. The modular prosthesis includes an intramedullary rod 12 which provides stability, a body element 18 which provides load transfer and rigidity, and a neck element 48 which provides a protuberance for mounting the head element 68. The rod has a distal end 12 and a proximal end 14 (FIG. 4), the proximal end of the rod preferably having a smaller diameter than the distal end. The distal end 12 is inserted into the patient's humerus and forms the stabilizing connection for maintaining the prosthesis in alignment with the axis of the humerus. The distal end of the rod may have flutes 16 (FIG. 19) to increase the surface area of the junction between the rod and the intramedullary canal of the humerus to add rotational stability to the prosthesis. The distal end of the rod may also have a slot (not shown) along the longitudinal axis of the rod to better accommodate the internal anomalies occurring in the interior of the intramedullary canal. This structure allows the distal end of the rod to compress to a smaller diameter to more easily reach the desired depth of insertion.

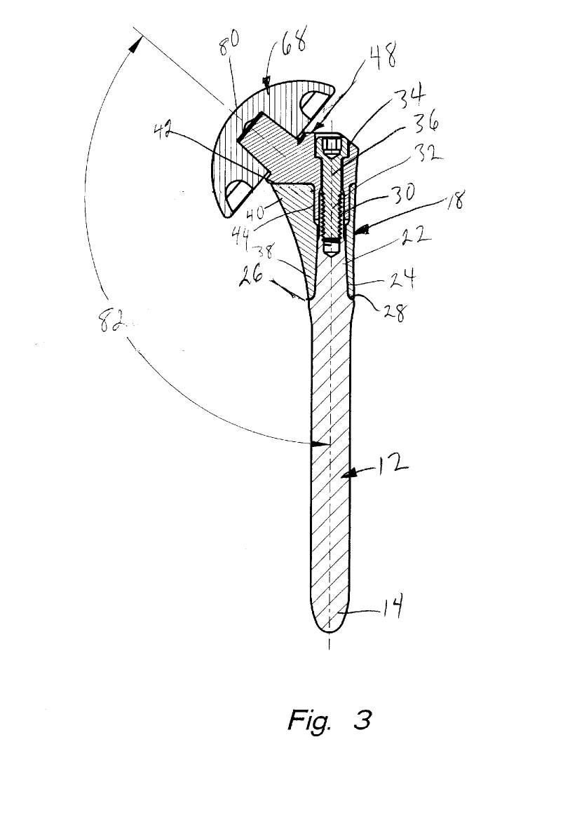

The body element 18 is mounted on the proximal end of the intramedullary rod. The body element has a through bore portion 20, through which the proximal end 14 of the intramedullary rod is inserted. As shown in FIG. 3, the through bore portion 20 and the proximal end 14 of the intramedullary rod have corresponding self-holding tapered surfaces 22, 24 which allow rotational movement between the components until a drawing force is applied to the rod, locking the elements together preventing any substantial rotational movement. In this manner, orientation of the body 18 with respect to the rod 12 can be established after insertion of the rod into the humerus. As the two elements are telescoped together, the tapered walls engage each other further strengthening the connection between the elements. In the preferred embodiment, the intramedullary rod has a shoulder 26 engaging the distal circumference of the body 18 for additional support. This additional support is desired when the body 18 is formed of an interconnecting cellular structure to promote bone ingrowth. It should also be noted that any of the components of the prosthesis may include a coating of on-growth material. Such on-growth materials may include, but should not be limited to, HA coating, Ti Plasma Spray and the like. Either the cooperating tapered surfaces 22 and 24 or the shoulder 26 and seating face 28 establish a precise limit to the distance the body may be telescoped over the intramedullary rod. This limit, in turn, establishes the overall length of the two elements. In addition, the proximal end of the intramedullary rod may include a stem 70 constructed and arranged to cooperate with an internal bore of the neck element 48 to provide additional stability to the assembly. The stem includes a pilot portion 74 having a cylindrical diameter and a threaded bore 30 for receiving the threaded end of bolt 32. The proximal end of the neck 48 has a counterbore portion 34 which is constructed and arranged to accept the head of the fastener 36. This counterbore portion 34 may be cylindrical or conical. If conical, the walls of the counterbore portion 34 taper from a large diameter proximal end toward the distal end. In an alternative embodiment, shown in FIGS. 24-25, the proximate end of the intramedullary rod has a threaded stem 92 for receiving a threaded nut 94. In this embodiment the counterbore is sized to accept at least a portion of the nut 94. The nut may include a pilot diameter 96 sized to engage the surface of the neck bore 98 to add rigidity to the assembly. The rod may be provided in various diameters and lengths, while the body may be provided in various fill sizes and heights, to allow assembly of a customized prosthesis.

In a most preferred embodiment the self-holding taper is a morse type taper, however it should be noted that other self-holding tapers well known in the art such as the American National, Jacobs, Jarno, Brown and Sharp, British Standard and suitable combinations thereof may be utilized without departing from the scope of the invention.

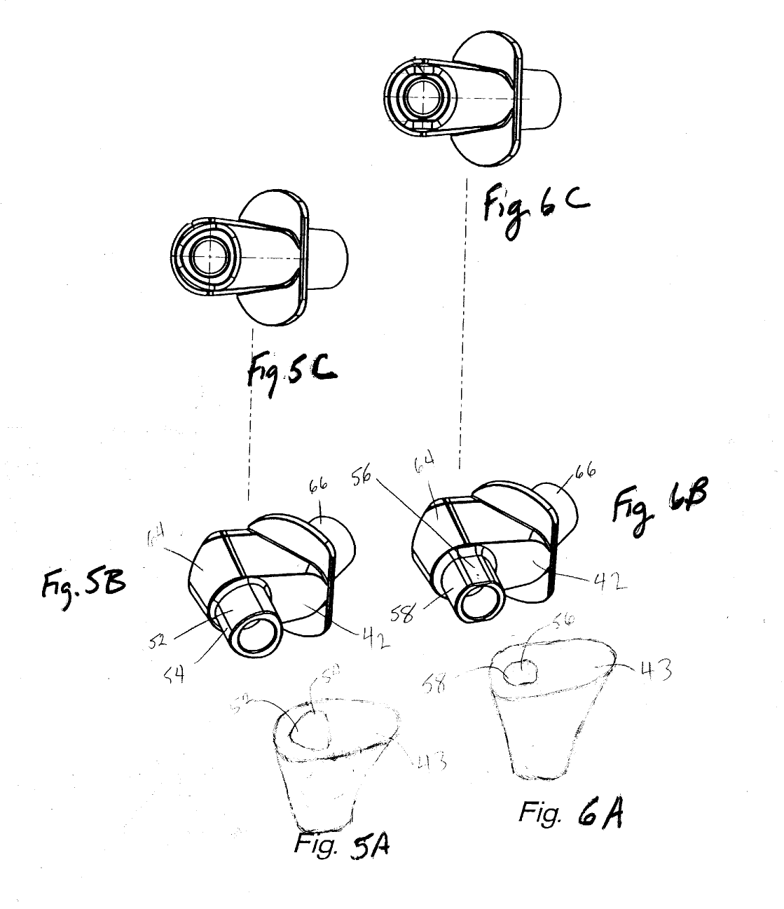

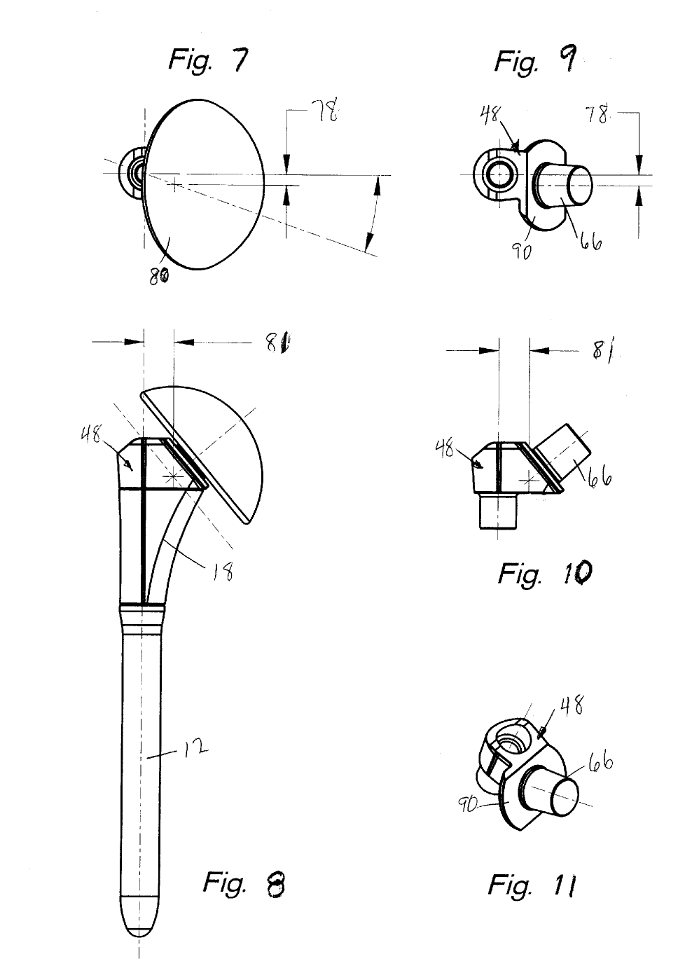

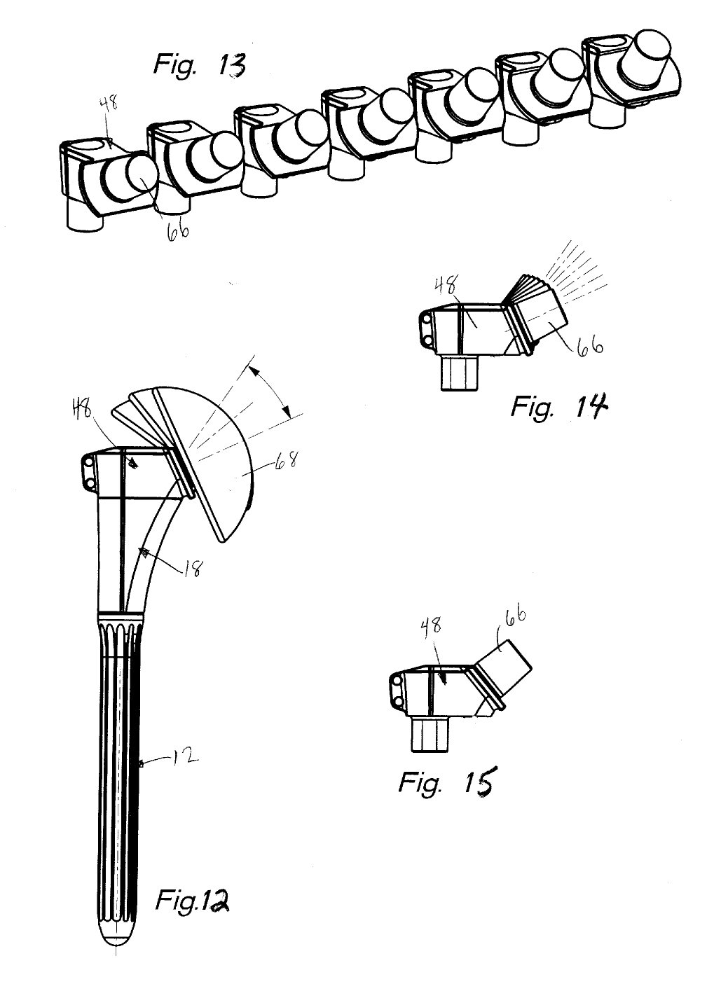

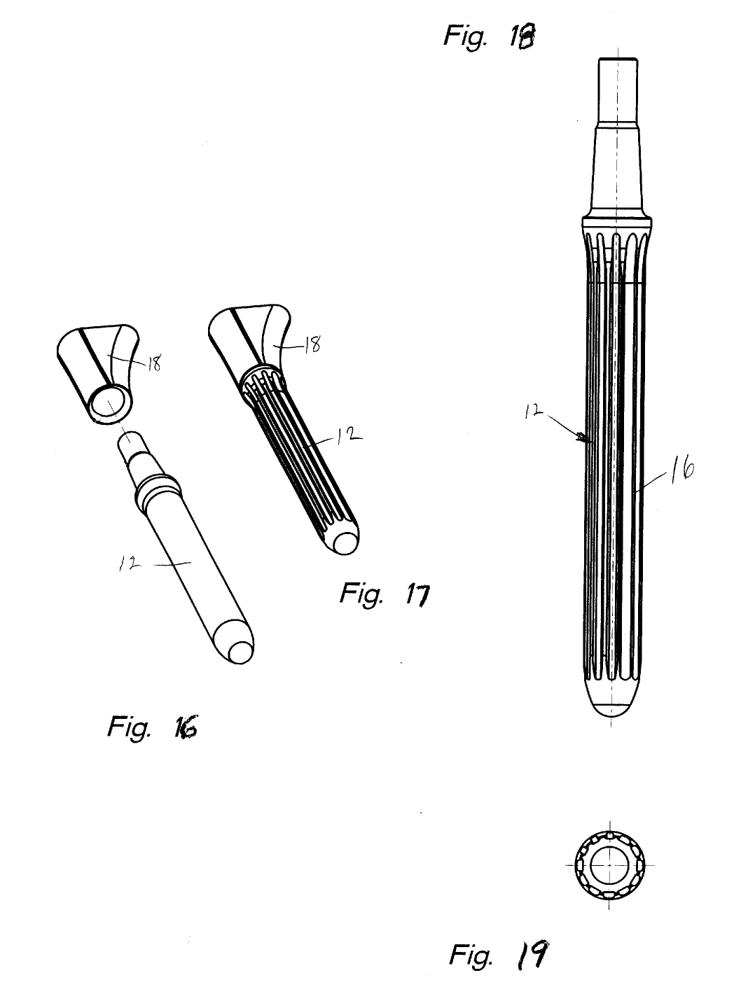

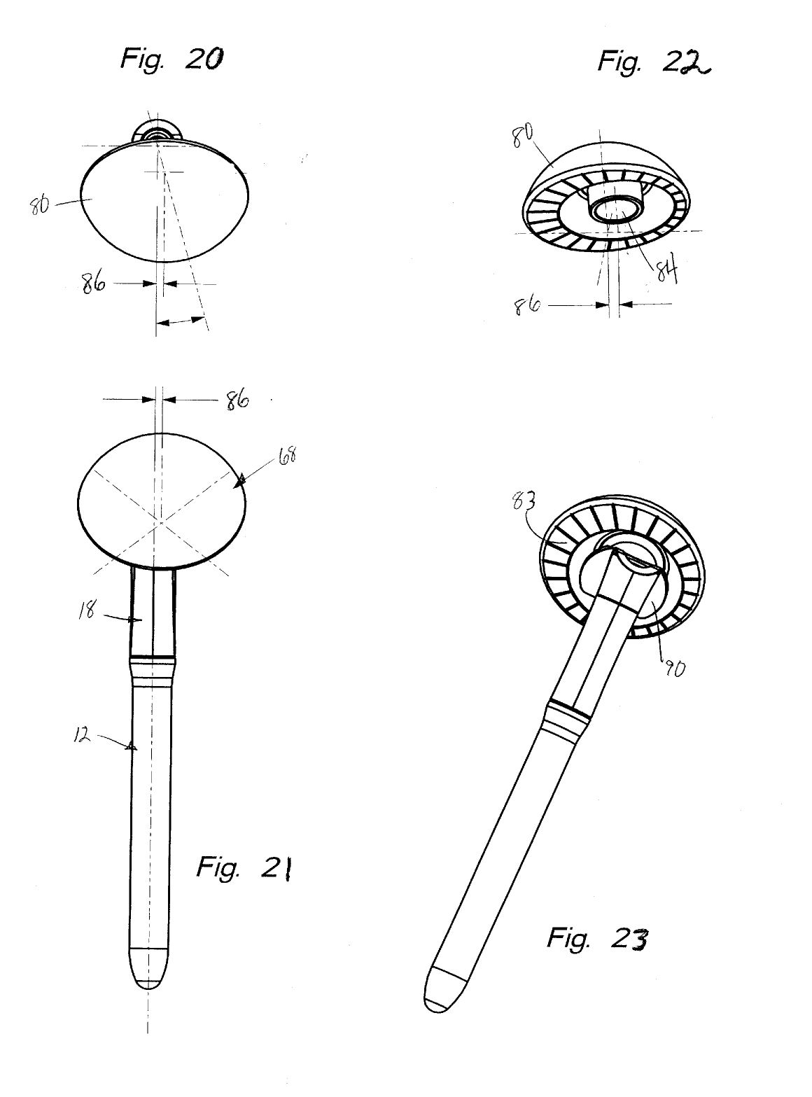

Once inserted, the intramedullary rod 12 provides stability and the body 18 acts as the load bearing element for the articular head 68. The body 18 is shaped like the natural humerus head and has an outer diameter that is preferably larger than the intramedullary rod 12 at the distal end. The distal end 38 of the body 18 is inserted into the intramedullary canal. This junction of the body and the shaft of the femur is the primary load carrying connection between the prosthesis and the patient's body. The body flares to a larger diameter proximal end 40 which has a substantially planar load-bearing surface 42 containing a contoured interlocking socket 44 that is constructed and arranged to cooperate with the neck element 48. As shown in FIGS. 5 and 6, the socket 44 and first protuberance include substantially conjugate interlocking surfaces. In one embodiment the first protrusion and the first socket each include at least one and preferably two planar surfaces 56 connected by two curved surfaces 58 as illustrated in FIG. 6. In an alternative embodiment the first protrusion and the first socket each include three first curved segments 52, having a first radius, connected by three second curved segments 54, having a second radius. In either embodiment the first neck protrusion and said body socket are constructed and arranged to be telescoped together in a slip-fit manner with the planar surfaces and curved surfaces in intimate contact with each and the load-bearing surfaces in contact with each other. The cooperation between the protrusion and the socket provide axial alignment between the two components and the interlocking contoured shapes prevent rotational movement between the components. While the load-bearing surfaces 42, 43 distribute loads from the head 68 across a broader area of the rod to provide rigidity, strength and stability to the prosthesis. It should also be noted that other contoured interlocking protrusion and socket arrangements and shapes, not shown, may be utilized without departing from the scope of the invention.

Referring generally to the Figs., the neck 48 has a partially cylindrical body 64 with a laterally extending second protrusion 66 having a self-holding tapered shape for fixable engagement with a head element 68. This protrusion 66 carries the head element for an artificial shoulder and can be specifically set at different inclination and retroversion angles, as shown in FIGS. 8-15 with respect to the body element 18 and thus the axis of the humerus. In one embodiment, illustrated in FIGS. 8-12 the first protrusion may be constructed with a preset posterior offset 78 and/or medial offset 81. The medial offset may be in a positive or negative direction to provide for left and right prosthesis. In addition, as illustrated in FIGS. 13-15, the first protrusion may be constructed to include various angles of inclination 82 for close approximations of the excised humerus head being replaced. FIG. 14 illustrates several non-limiting embodiments of the neck having various angles of inclination ranging from 115° to 145°. Other angles of inclination are also contemplated within the scope of the invention. The load-bearing surface of the neck 42 is preferably formed as a flat surface with a depending first protrusion 72. It should be noted, that while the protrusion is illustrated as protruding from the neck element and the socket is illustrated as extending inwardly into the body element, this organization could be reversed without departing from the scope of the invention.

One embodiment of the prosthesis is assembled by turning the threads of the bolt 36 into the threads of the intramedullary rod 12. As these cooperating screw threads tighten, the elements of the prosthesis are drawn together forcing the load bearing surfaces into intimate contact and forcing the tapered proximal end of the rod into a friction fit with the tapered bore of the body and the body to a stop limit on the intramedullary rod. In the final disposition, the body and the intramedullary rod are locked together over a major part of the length of each and the neck is locked to the rotationally immovable body at a specific retroversion angle by the first protrusion and socket arrangement.

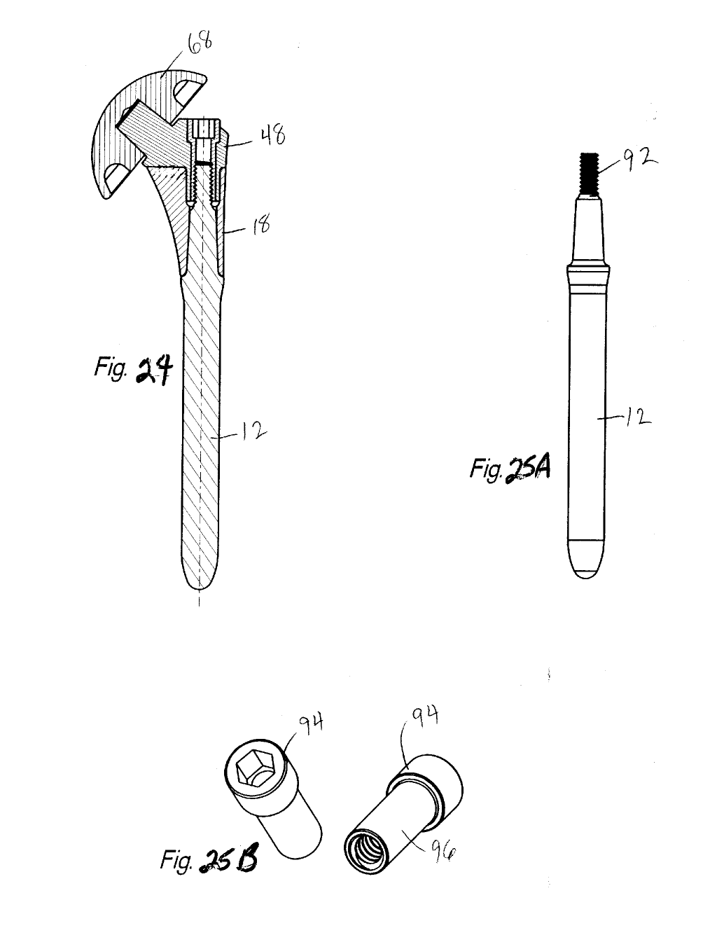

Referring to FIGS. 3 and 4, the preferred embodiment of the head is illustrated. The head includes an outer articulation surface 80 and an inner surface 83. The inner surface includes a socket 84 positioned substantially in the central portion of the head. The socket is constructed and arranged to cooperate with the second neck protrusion 66 and in a most preferred embodiment includes a self-holding tapered shape. In an alternative embodiment illustrated in FIGS. 21-24, the socket is constructed to be off-center a pre-determined distance 86 with respect to the articulation surface. The construction allows the head to be rotated to provide posterior offset to the articulation surface prior to locking the head in place. The head may be secured to the second protrusion with clamps using the outwardly extending tabs 90 or alternatively by tapping the head onto the tapered surface to engage the self-holding tapers.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A fixable modular prosthesis to be used in shoulder joint replacement comprising:
a rod having a distal end adapted for insertion into the intramedullary canal of a humerus bone and a proximal end including a self-holding taper for fixable coupling with a body element, said proximal end having a threaded blind bore substantially along a longitudinal axis of said intramedullary rod;
a body element having a narrow distal end and a larger proximal end, a through bore extending from said proximal end to said distal end, said distal end of said bore including a self-holding taper for fixable coupling to said proximal end of said rod, said proximal end of said body including a load-bearing surface constructed and arranged to cooperate with neck element, said load-bearing surface including a first socket for fixed non-rotational engagement with a neck element;
a neck having a load-bearing surface at a distal end, a first protrusion extending downwardly from said load-bearing surface, said first protrusion adapted for insertion into said first socket for non-rotational engagement therebetween, a proximal end having a second protrusion extending outwardly therefrom, said second protrusion extending at an obtuse angle of inclination with respect to said first protrusion, said second protrusion having a self-holding tapered shape for fixable engagement with a head element, a second bore extending through said neck, said second bore extending through said first protrusion to align with said blind bore;
a head element having a substantially spherical outer surface and an inner surface having a second socket, said second socket constructed and arranged to include a self-locking taper for fixable engagement with said second protrusion;
a threaded fastener having sufficient length to extend through said bore and said second bore for cooperation with said blind bore, whereby rotational movement between said rod and said body is facilitated prior to tightening of said fastener, whereby tightening of said fastener draws the elements of the prosthesis together forcing said load bearing surfaces into a juxtaposed position with respect to each other and forcing the tapered proximal end of the rod into a friction fit with the tapered bore of the body fixing the orientation of said prosthesis.

2. The modular prosthesis of claim 1 wherein said first neck protrusion includes at least one planar surface, the edges of said at least one planer surface connected by a curved surface, said proximal end of said body socket including at least one planar surface, the edges of said at least one planer surface connected by a curved surface, said first neck protrusion and said body socket constructed and arranged to be telescoped together with said planar surfaces and said curved surfaces in juxtaposed contact with each other, whereby rotation of said neck with respect to said body is prevented.

3. The modular prosthesis of claim 1 wherein said first protrusion of said neck includes opposite planar surfaces connected by curved surfaces, said body socket having a substantially conjugate shape, said first neck protrusion and said body socket constructed and arranged to be telescoped together with said planar surfaces and said curved surfaces in contact with each other, whereby rotation of said neck with respect to said body is prevented.

4. The modular prosthesis of claim 1 wherein said first protrusion of said neck includes three first curved segments, having a first radius, connected by three second curved segments, having a second radius, said body socket including a substantially conjugate shaped bore, whereby said first neck protrusion and said body socket are constructed and arranged to be telescoped together with said curved surfaces in intimate contact with surfaces within said socket, whereby rotation of said neck with respect to said body is prevented.

5. The modular prosthesis of claim 1 wherein said self-holding tapers are selected from the group consisting of American National, Jacobs, Jarno, Brown and Sharp, Morse, British Standard.

6. The modular prosthesis of claim 1 wherein said second protrusion of said neck element includes a posterior offset, wherein said medial offset is constructed and arranged to compensate for retroversion angle within a human anatomy.

7. The modular prosthesis of claim 1 wherein said angle of inclination is at least about one hundred and five degrees and less than one hundred and fifty degrees.

8. The modular prosthesis of claim 1 wherein said distal end of said rod includes a fluted surface for anti-rotational stability.

9. The modular prosthesis of claim 1 wherein said second socket of said head element is offset from the central portion of said head, whereby said head may be rotated with respect to said second protrusion for retroversion compensation prior to locking engagement with said second protrusion.

10. The modular prosthesis of claim 1 wherein at least one of said modular prosthesis components are coated with a bone growth material.

11. The modular prosthesis of claim 1 wherein a plurality of different sized intramedullary rods and bodies as well as a plurality of necks having different angles of inclination, and at least one head are provided in a kit, said components being constructed and arranged to interchangeably cooperate in any combination to assemble a prosthesis with specific characteristics.

12. A fixable modular prosthesis to be used in shoulder joint replacement comprising:
a rod having a distal end adapted for insertion into the intramedullary canal of a humerus bone and a proximal end including a self-holding taper for fixable coupling with a body element, said proximal end having a threaded stem substantially along a longitudinal axis of said intramedullary rod;
a body element having a narrow distal end and a larger proximal end, a through bore extending from said proximal end to said distal end, said distal end of said bore including a self-holding taper for fixable coupling to said proximal end of said rod, said proximal end of said bore including a first socket for fixed engagement with a neck element;
a neck having a flat surface at a distal end, a first protrusion extending downwardly from said flat surface, said first protrusion adapted for insertion into said first socket for non-rotational engagement therebetween, a proximal end having a second protrusion extending outwardly therefrom, said second protrusion extending at an obtuse angle of inclination with respect to said first protrusion, said second protrusion having a self-holding tapered shape for fixable engagement with a head element, a second bore extending through said neck, said second bore extending through said first protrusion to align with said blind bore;
a head element having a substantially spherical outer surface and an inner surface having a second socket, said second socket constructed and arranged to include a self-locking taper for fixable engagement with said second protrusion;
a nut having a threaded internal bore for cooperation with said threaded stem, whereby rotational movement between said rod and said body is facilitated prior to tightening of said nut, whereby rotational movement of said nut provides a drawing force to provide a prosthesis having a fixed orientation.

13. The modular prosthesis of claim 12 wherein said first neck protrusion includes at least one planar surface, the edges of said at least one planer surface connected by a curved surface, said proximal end of said body socket includes at least one planar surface, the edges of said at least one planer surface connected by a curved surface, said first neck protrusion and said body socket constructed and arranged to be telescoped together with said planar surfaces and said curved surfaces in intimate contact with each other, whereby rotation of said neck with respect to said body is prevented.

14. The modular prosthesis of claim 12 wherein said first protrusion of said neck includes opposite planar surfaces connected by curved surfaces, said body socket including opposite planar surfaces connected by curved surfaces, said first neck protrusion and said body socket constructed and arranged to be telescoped together with said planar surfaces and said curved surfaces in intimate contact with each other, whereby rotation of said neck with respect to said body is prevented.

15. The modular prosthesis of claim 12 wherein said first protrusion of said neck includes three first curved segments, having a first radius, connected by three second curved segments, having a second radius, said body socket including a substantially conjugate shaped bore, whereby said first neck protrusion and said body socket are constructed and arranged to be telescoped together with said curved surfaces in intimate contact with surfaces within said socket, whereby rotation of said neck with respect to said body is prevented.

16. The modular prosthesis of claim 14 wherein said second protrusion of said neck element includes a medial offset, wherein said medial offset is constructed and arranged to compensate for the hinge point between the proximal metaphyseal axis and the top of the articular head.

17. The modular prosthesis of claim 12 wherein a plurality of different sized intramedullary rods and bodies as well as a plurality of necks having different angles of inclination, and at least one head are provided in a kit, said components being constructed and arranged to interchangeably cooperate in any combination to assemble a prosthesis with specific characteristics.
